(19) United States
(12) Patent Application Publication
(10) Pub. No.: US 2001/0026796 A1
(43) Pub. Date: Oct. 4, 2001

(54) TCL1 enhances Akt kinase activity and mediates its nuclear translocation

(76) Inventors: Carlo M. Croce, Philadelphia, PA (US); Yuri Pekarsky, Philadelphia, PA (US)

Correspondence Address: CLIFFORD K. WEBER, ESQ, OFFICE OF UNIVERSITY COUNSEL, THOMAS JEFFERSON UNI, 1020 WALNUT STREET, SUITE 620, PHILADELPHIA, PA, 19107, US

(21) Appl. No.: 09/804,656

(22) Filed: Mar. 13, 2001

Related U.S. Application Data
(60) Provisional application No. 60/189,245, filed on Mar. 14, 2000.

(51) Int. Cl. A61K 38/45; G01N 33/574

(52) U.S. Cl. 424/94.5; 435/7.23

(57) ABSTRACT

The TCL1 oncogene at 14q32.1 is involved in the development of human leukemia. This invention demonstrates the interaction between the Tcl1 and the Akt1 proteins. The physical interaction between endogenous Akt1 and Tcl1 occurs through the PH domain of the Akt1 protein. The present invention relates to the identification of Tcl1 mimics and Tcl1 antagonists that modifying this interaction, with the subsequent modification of apoptotic and proliferative signals.

### CROSS REFERENCE TO RELATED APPLICATIONS

This invention claims priority under 35 U.S.C. §119 based upon U.S. Provisional Patent Application No. 60/189,245 filed Mar. 14, 2000.

### GOVERNMENT RIGHTS TO THE INVENTION

This invention was made in part with government support under Grant numbers CA76259 and RO1CA57436 awarded by the National Institutes of Health. The government has certain rights to the invention.

### FIELD OF THE INVENTION

The present invention generally relates to the field of molecular biology, more particularly to the interaction between the two oncogene products Tcl1 and Akt1, modification of this interaction and the subsequent modification of apoptotic and proliferative signals.

### BACKGROUND OF THE INVENTION

The TCL1 gene at chromosome 14q32.1 is often activated in human T-cell malignancies by chromosomal inversions and translocations such as inv(14)(q11;q32) and t(14;14)(q11;q32) or t(7;14)(q35;q32). (Virgilio, L., et al., Proc. Natl. Acad. Sci. USA 91:12530-12534, 1994). Normally TCL1 expression is observed in early T-cell progenitors (CD4-, CD8-, CD3-), in pre B-cells, and immature IgM expressing B-cells. (Virgilio, L., et al., Proc. Natl. Acad. Sci. USA 91:12530-12534, 1994). Introduction of a TCL1 transgene into mice under the control of the proximal Ick promoter resulted in mature T-cell leukemia in mice at the age of 15 to 20 months. (Virgilio, L., et al., Proc. Natl. Acad. Sci. USA 95:3885-3889, 1998). The second member of the TCL1 gene family, MTCP1, is located at Xq28 and activated in rare cases of mature T-cell leukemia showing rearrangements at Xq28. (Soulier, J., et al., Oncogene 9:3565-3570, 1994). Recently the third member of this family was identified, TCL1b, and found to also be located at 14q32.1 and activated by chromosomal rearrangements involving the TCL1 locus. (Pekarsky, Y., et al., Proc. Natl. Acad. Sci. USA 96:2949-2951, 1999). In the mouse, Tcl1b is represented by five homologues. (Hallas, C., et al., Proc. Natl. Acad. Sci. USA 96:14418-14423, 1999). Although the crystal structure of Tcl1 suggests that it plays a role in the transport of small molecules such as retinoids, nucleotides and fatty acids (Fu, Z. Q., et al., Proc. Natl. Acad. Sci. USA 95:3413-3418, 1998), the function of the 14 kD Tcl1 protein is still not known. Cell fractionation experiments in lymphoid cells have shown that Tcl1 is localized in both the nucleus and the cytoplasm. (Fu, T. B., et al., Cancer Res. 54:6297-6301, 1994).

The protein kinase Akt/PKB is the homologue of v-akt, isolated from the retrovirus AKT8, which causes T-cell lymphomas in mice. (Bellacosa, A., et al., Science 254:274-277, 1991). The Akt protein contains a pleckstrin homology (PH) domain and kinase domain. (Chan, T. O., et al., Annu. Rev. Biochem. 68:965-1014, 1999). Activation of Akt by insulin and various growth and survival factors involves a PI-3 kinase-dependent membrane translocation step which is due to the binding of the PH domain to D3 phosphoinositides; and a PDK1-mediated phosphorylation step at Thr308 and Ser473. (Chan, T. O., et al., Annu. Rev. Biochem. 68:965-1014, 1999). Treatment with wortmannin, a PI3-kinase inhibitor, completely inhibits the activation of Akt. (Chan, T. O., et al., Annu. Rev. Biochem. 68:965-1014, 1999). Recent studies showed that Akt is a key player in the transduction of antiapoptotic and proliferative signals in T-cells. (Ahmed, N. N., et al., Proc. Natl. Acad. Sci. USA 94:3627-3632, 1997; Mok, C. L., et al., J. Exp. Med. 189:575-86, 1999; Chan, T. O., et al., Annu. Rev. Biochem. 68:965-1014, 1999). Activated Akt enhances both cell cycle progression and IL2 production, and thus inhibition of the proapoptotic factor Bad. (Mok, C. L., et al., J. Exp. Med. 189:575-86, 1999). Introduction of a constitutively activated AKT1 transgene under the control of the proximal Ick promoter causes T-cell lymphomas in mice (Malstrom, S. and Tsichlis, P. N. Unpublished data). In cultured cells Akt1 is localized in both the nucleus and cytoplasm. (Ahmed, N. N., et al., Oncogene 8:1957-63, 1993). In addition, it has been claimed that Akt1 translocates to the nucleus in insulin stimulated 293 cells. (Andjelkovic, M., et al., J. Biol. Chem. 272:31515-31524, 1997). The mechanism of the nuclear translocation of Akt1 is not known.

The present invention provides evidence that Tcl1 and Akt1, the protein products of two oncogenes involved in T-cell leukemogenesis, interact with each other. This interaction is mediated by the PH domain of Akt1 and results in enhancement of the Akt1 kinase activity, as well as promoting the nuclear translocation of the Akt1 kinase. The present invention further relates to inhibiting the interaction between Tcl1 and Akt1, thereby inhibiting any aberrant Akt1 induced proliferative signals in T-cells.

### DEFINITIONS

“modulate” means to inhibit or down-regulate or restrain the activity

“modify” means to change the activity from that which is endogenous

“antagonist” means to oppose the action of

### SUMMARY OF THE INVENTION

It is an object of the present invention to provide an antibody which binds to an epitope on Tcl1, this antibody will modulate the interaction between Tcl1 and Akt1 kinase. It is a further object of the invention for the antibody to modulate the Tcl1 enhanced kinase activity of the Akt1 kinase. In one embodiment of the invention the antibody is a monoclonal antibody. In another of the invention the antibody is a polyclonal antibody.

It is another object of the present invention to provide a pharmaceutical composition containing an antibody that binds to an epitope on Tcl1 so as to modulate the interaction between the Tcl1 and the Akt1 kinase.

The present invention also provides a method of treating a disease state in which the activity of Akt1 kinase is altered in a mammal. Administration of a therapeutically effective amount of the antibody will allow for the antibody to bind to an epitope on Tcl1 and modulate the Tcl1 enhanced kinase activity of the Akt1 kinase. The disease state to be treated is a T-cell leukemia or T-cell lymphoma. In one embodiment of the invention the T-cell leukemia or T-cell lymphoma is associated with a chromosome 14 abnormality. In another embodiment this chromosome 14 abnormality is a t(14;14) (q11;q32) translocation or an inv (14) (q11;132) inversion.

The present invention further provides a method of treating a disease state in which the activity of an Akt1 kinase is altered in a mammal by administration of a therapeutically effective amount of a peptide fragment of Akt1 kinase. In one embodiment of the invention the peptide fragment is the PH domain of the Akt kinase. Binding of the peptide fragment or the PH domain fragment will modulate the Tcl1 enhanced kinase activity of the Akt1 kinase. The disease state to be treated is a T-cell leukemia or T-cell lymphoma. In one embodiment the T-cell leukemia or T-cell lymphoma is associated with a chromosome 14 abnormality. In another embodiment of the present invention the chromosome 14 abnormality is a t(14;14) (q11;q32) translocation or an inv (14) (q11;132) inversion.

The present invention further provides a method of treating a disease state wherein the PH domain fragment of Akt1 kinase competitively binds to the Akt1 binding domain on the Tcl1 protein.

It is also an object of the present invention to provide a pharmaceutical composition containing a peptide fragment of Akt1 kinase or the PH domain fragment of Akt1 kinase.

The present invention further provides a compound that is a Tcl1 mimic which binds to Akt1 kinase in any cell and is functionally active in mimicking the Tcl1 enhanced activation of the Akt1 kinase.

It is another object of the present invention to provide a method for identifying a molecule that specifically binds to Atk1 kinase and is functionally active in mimicking the Tcl1 enhanced activation of the Akt1 kinase. The Akt1 kinase is brought into contact with a plurality of molecules under conditions that are conducive to binding between the Akt1 kinase and the molecules. Molecules which specifically bind to the Akt1 kinase, and are functionally active in mimicking the Tcl1 enhanced activation, are thereby identified.

The present invention provides a method of treating a disease state in which the activity of Akt1 kinase is altered in a mammal. Administration of a therapeutically effective amount of the Tcl1 mimic will allow for the Tcl1 mimic to bind to the Akt1 kinase and activate the Tcl1 enhanced kinase activity of the Akt1 kinase. In one embodiment of the invention the disease state is a degenerative disease.

The present invention further provides a pharmaceutical composition containing a Tcl1 mimic which will activate the Tcl1 enhanced kinase activity of the Akt1 kinase.

It is another object of the present invention to provide a compound that is a Tcl1 antagonist that binds to the Akt1 kinase in any cell and is functionally active in modulating the Tcl1 enhanced activation of the Akt1 kinase.

The present invention also provides a method for identifying a molecule that specifically binds to Atk1 kinase and is functionally active in antagonizing the Tcl1 enhanced activation of the Akt1 kinase. The Akt1 kinase is brought into contact with a plurality of molecules under conditions conducive to binding between the Akt1 kinase and the molecules. Molecules that specifically bind to the Akt1 kinase and are functionally active in antagonizing the Tcl1 enhanced activation are thereby identified.

It is a further object of the present invention to provide a method of treating a disease state in which the activity of Akt1 kinase is altered in a mammal. A therapeutically effective amount of a Tcl1 antagonist is administered to the mammal so that the Tcl1 antagonist binds to the Akt1 kinase, thereby inhibiting the Tcl1 enhanced kinase activity of the Akt1 kinase. In one embodiment of the invention disease state is a proliferative disorder.

The present invention further provides a pharmaceutical composition containing a Tcl1 antagonist that inhibits the Tcl1 enhanced kinase activity of Akt1 kinase.

### DESCRIPTION OF THE DRAWINGS

FIG. 1. Tcl1 interacts with Akt. A. Immunoprecipitation of Akt1 with anti-Tcl1 antibody. Detection of Akt1 in the immunoprecipitates was carried out by Western blotting using a mouse monoclonal anti-Akt1 antibody. Lysates used: Lanes 1-3, 293 cells transfected with TCL1; Lanes 4-6, SupT11 cells. Antibodies used for immunoprecipitation: anti-Tcl1 (lanes 1 and 4), mouse IgG (lanes 2 and 5), mouse monoclonal anti-Akt1 (lanes 3 and 6). B. Akt1 interacts with Tcl1 through PH domain. 293 cells were cotransfected with TCL1 and HA-AKT1 or HA-(Δ11-60) AKT1 mutant as indicated. Immunoprecipitations were carried out with an anti-HA antibody (lanes 1 and 2), mouse IgG (lanes 3 and 4), or anti-Tcl1 antibody (lanes 5 and 6) and detected by Western blotting with anti-Tcl1 antibody. Lanes 7 and 8, The lysate was coprecipitated with 5 μg of Akt1 PH domain-GST fusion protein (lane 7) or GST alone (lane 8). C. Akt1, but not Akt2 strongly interacts with Tcl1. 293 cells were cotransfected with TCL1 and HA-AKT1 (lanes 1-3) or HA-AKT2 (lanes 4-6). IPs were carried out with anti-Tcl1 antibody (lanes 1 and 4), mouse IgG (lanes 2 and 5), and anti-HA antibody (lanes 3 and 6) and detected with anti-Tcl1 antibody. D. Interaction with Tcl1 is independent of Akt1 phosphorylation. 293 cells were cotransfected with TCL1 and HA-AKT1 (lanes 1-3) or HA-AKT1 AA mutant (Thr308/Ala Ser473/Ala). IPs and Western blot detection were performed as in C. Expression levels of exogenous and endogenous Tcl1 and Akt were checked in each experiment (where applicable) and were found similar.

FIG. 2. Tcl1 enhances Akt1 kinase activity. Endogenous Akt1 was immunoprecipitated from 293 cells transfected with the indicated constructs. Kinase activity was determined using GSK3-β-GST fusion protein as a substrate. Each reaction was terminated after 0, 4, 10, and 30 minutes Amount of Akt (top panel) and phospho-GSK3-β (lower panel) were determined by Western blotting with rabbit anti-Akt antibody and anti-phospho-GSK3-β antibody, respectively. A. Akt1 was immunoprecipitated from TCL1 transfected cells with an anti-Tcl1 antibody (left) or vector transfected cells with anti-Akt antibody (right). B. Same lysates as in A, but immunoprecipitations were carried out with an anti-Akt antibody, only. C. Lysates of thymus from a TCL1-transgenic mouse (2) (left) or a wild-type mouse (right) were immunoprecipitated with anti-Akt antibody. For immunoprecipitation of Akt anti-PKBα/Akt1 clone 7 antibody was used, anti-Akt/PKB rabbit polyclonal antibody or anti-Akt antibody included with Akt kinase assay kit was used with consistent results.

FIG. 3. The expression of Tcl1 does not increase Akt1 phosphorylation or interfere with effect of wortmannin. NIH-3T3 cells were transfected with TCL1 (lanes 1,3,5,7) or vector (lanes 2,4,6,8) and starved with media without FCS overnight. A. Cells were treated with 100 ng/ml PDGF for the indicated period of time and lysed. Western blotting was performed using anti-phospho-Akt and anti-Tcl1 antibody. Each lane contains the same amount of protein. B. NIH-3T3 were transfected and starved as in A. Cells were not treated (lanes 1 and 2); treated with 200 nM wortmannin for 1,5 hours (lanes 3 and 4); treated with 100 ng/ml PDGF for 30 minutes (lanes 5 and 6); treated with 200 nM wortmannin for 1 hour followed by PDGF for 30 min (lanes 7 and 8). Western blotting was performed as in A.

FIG. 4. Tcl1 promotes nuclear translocation of Akt1. MEF cells were transfected or cotransfected with indicated constructs. A. Intracellular localization of Akt1 (left), Tcl1 (middle), and GFP-Tcl1 (right). B. Colocalization of Akt1 (green) and Tcl1 (red). C. Co-localization of Akt1 (red) and GFP-Tcl1 (green). D. Intracellular localization of Akt1 (red) and Tcl1-GFP (green).

FIG. 5. Nuclear translocation of Akt1 by Tcl1 requires their interaction in the cytoplasm. MEF cells were transfected or cotransfected with indicated constructs. A. Intracellular localization of Akt1 (red) and nucTcl1 (green) in the same cells. B. Intracellular localization of myristoylated Akt1 (green) and Tcl1 (red) in the same cells.

### DESCRIPTION OF THE INVENTION

Materials and Methods.

Cells Lines

293 and NIH-3T3 cells were purchased from the American Type Culture Collection (Rockville, Md.). MEF cells were obtained from Clontech (Palo Alto, Calif.). SupT11 T-cell leukemia cells were described in (Virgilio, L., et al., Proc. Natl. Acad. Sci. USA 91:12530-12534, 1994), which is incorporated herein by reference.

Constructs and Transfection

HA-AKT1, (Δ11-60)-HA-AKT1, (Thr308/Ala, Ser473/Ala)-HA-AKT1, (Lys179/Met)-HA-AKT1 and HA-AKT2 constructs were previously described. (Bellacosa, A., et al., Oncogene 17:313-325, 1998). All HA-AKT constructs contain murine Akt1 or Akt2 ORF and the HA tag on the N-terminus of an encoded protein. The myristoylated Myc-AKT1 contruct and Akt1 PH domain GST fusion protein were purchased from Upstate Biotechnology (Lake Placid, N.Y.). Full length TCL1 cDNA was amplified by PCR from SupT11 mRNA and cloned into pcDNA3, pCMV/myc/nuc vectors (Invitrogen, Carlsbad, Calif.), and into pEGFPN1 and pEGFPC1 vectors (Clontech). Transfections were carried out using Fugene 6 reagent (Roche, Indianapolis, Ind.) according to the manufacturer's instructions.

Protein Lysates, Immunoprecipitation, and Western Blotting

Cells were grown in RPMI-1640 or MEM medium with 10% FCS and lysed using NP40 lysis buffer containing 50 mM Tris (pH7.5), 150 mM NaCl, 10% Glycerol, 0.5% NP40, and protease inhibitors. Immunoprecipitations were carried out overnight in the same buffer using 0.5 mg of protein, 5 μg of antibody, and 40 μl of protein AIG PLUS agarose (Santa Cruz Biotechnology, Santa Cruz, Calif.) and washed 4 times with the same buffer containing 0.1% NP40. Antibodies used were: Anti-HA.11 (BAbCO, Richmond, Calif.), anti-PKBα/Akt clone 7 (Transduction Laboratories, San Diego, Calif.), or anti-Akt/PKB rabbit polyclonal antibody (New England Biolabs, Beverly, Mass.), anti-phospho-Akt(Ser 473) rabbit polyclonal antibody (New England Biolabs), and anti-Tcl1 clone 27D6 mouse monoclonal antibody. Western blotting was performed under standard conditions. (Fu, T. B., et al., Cancer Res. 54:6297-6301, 1994).

Kinase Assay

These experiments were carried out using the Akt kinase assay kit from New England Biolabs according to the manufacturer's recommendations; in some experiments anti-Tcl1 or anti-HA antibodies were used for immunoprecipitations.

Immunofluorescence

Cells were seeded on fibronectin covered cell culture slides (Becton Dickinson Labware, Bedford, Mass.), fixed for 10 minutes in 3.7% PBS buffered formaldehyde and permeabilized with 0.05% Triton X100 in PBS for 5 minutes Cells were then blocked for 1 hour in 100% goat serum (Sigma, St. Louis, Mo.), incubated with a primary antibody for 1 hour in 10% goat serum in PBS and with a secondary antibody under the same conditions. Antibodies used were: anti-Tcl1 clone 27D6 mouse monoclonal antibody, anti-PKBα/Akt1 clone 7, rabbit anti-Akt antibody, anti-Myc rabbit polyclonal antibody (Upstate Biotechnology), anti-mouse Texas Red conjugated antibody (Oncogene Research products, Cambridge, Mass.) and anti-rabbit FITS conjugated antibody (Amersham, Piscataway, N.J.). Cells were examined using confocal microscope (Bio Rad, Hercules, Calif.) under 63× magnification.

Results

Tcl1 interacts with Akt1

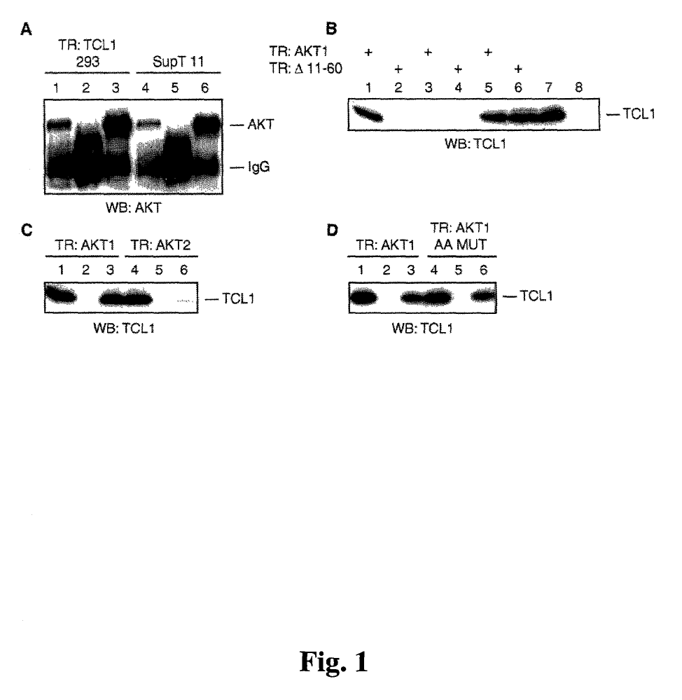

To determine if Tcl1 and Akt1 function in the same pathway the physical interaction between Tcl1 and Akt1 was analyzed. Immunoprecipitation with anti-Tcl1 antibodies followed by Western blotting with the monoclonal anti-Akt1 antibody revealed that Tcl1 interacts with endogenous Akt1 when transfected into 293 embryonic kidney cells (FIG. 1a, lanes 1-3). Endogenous Tcl1 and Akt1 also interact in SupT11 T-cell leukemia cells carrying a t(14;14)(q11;q32.1) translocation (FIG. 1a, lanes 4-6).

The Akt PH domain functions both as a phosphoinositide and as a protein binding module (Chan, T. O., et al., Annu. Rev. Biochem. 68:965-1014, 1999), therefore the involvement of the Akt PH domain in the Akt1/Tcl1 interaction was analyzed. The 293 cells were cotransfected with a TCL1 construct and HA-tagged AKT1 constructs expressing the wild type Akt1 protein or an Akt1 mutant protein (Akt1 A11-60), carrying a 50 amino acid PH domain deletion. The Akt1 was immunoprecipitated with the anti-HA antibody. Western blots of the immunoprecipitates were probed with the anti-Tcl1 antibody. FIG. 1b shows that Tcl1 interacts with wild type Akt1, but not with Akt1 (Δ11-60) (lanes 1 and 2). To prove that the PH domain is indeed responsible for this interaction an Akt1 PH domain GST fusion protein was used in pulldown experiments. FIG. 1b (lanes 7 and 8) shows that Tcl1 binds to the PH domain GST fusion protein, but not to GST alone.

The anti-Akt1 antibody used in FIG. 1a recognizes both Akt1 and Akt2, therefore a determination as to which isoform(s) of Akt actually interacts with Tcl1 was made. 293 cells were transfected with HA-tagged constructs of AKT1 or AKT2 in combination with the TCL1 construct. Lysates of the transfected cells were subjected to immunoprecipitation with an anti-HA antibody. FIG. 1c shows that Tcl1 strongly interacts with Akt1, since almost as much Tcl1 was precipitated with the anti-HA antibody as with the anti-Tcl1 antibody. In contrast, only a faint band of Tcl1 was observed in the Akt2 immunoprecipitates, even after prolonged exposures, implying that Tcl1 has a much stronger affinity for Akt1 than Akt2. Immunoprecipitation of Tcl1 also led to the coimmunoprecipitation of Akt1 (FIG. 1a), but not Akt2. Human Tcl1b did not coimmunoprecipitate with Akt1 or Akt2.

Tcl1 Enhances the Akt1 Kinase Activity

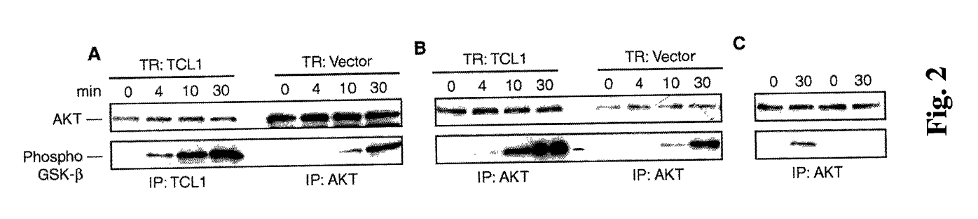

To determine whether Tcl1 affects the kinase activity of Akt1, 293 cells were transfected with a TCL1 expression construct or vector only. Endogenous Akt1 was immunoprecipitated 48 hours later from lysates of the transfected cells using anti-Tcl1 or anti-Akt1 antibodies. The kinase activity associated with these immune complexes was measured using a GST-GSK3-β fusion protein as a specific substrate. FIG. 2a shows that the specific activity of Tcl1-bound Akt1 immunoprecipitated from TCL1 transfected cells is 5-10 times higher than the specific activity of Akt1 immunoprecipitated from vector transfected cells (FIG. 2a). The specific activity of Akt1 immunoprecipitated with the anti-Akt1 antibody is also higher in Tcl1 transfected cells versus vector transfected cells (FIG. 2b). The more moderate increase of the kinase activity of Akt1 immunoprecipitated from Tcl1 transfected cells versus vector transfected cells in FIG. 2b versus FIG. 2a is due to the fact that only a fraction of Akt1 immunoprecipitated with the anti-Akt1 antibody is bound to Tcl1. Nevertheless, in both panels the activity of Akt1 is higher in Tcl1 transfected cells at 10 minutes of incubation (FIGS. 2a and 2b).

To verify that the kinase activity in the Akt1 immunoprecipitates is due to Akt1 and not another associated kinase, the activity of a kinase dead mutant of Akt1 (Lys179/Met) expressed under similar conditions was analyzed. As expected, immunoprecipitates of this mutant did not show any kinase activity. These findings were further confirmed by experiments showing that Akt1 is constitutively active in the thymus of transgenic mice expressing Tcl1 under the control of the proximal Ick promoter (Virgilio, L., et al., Proc. Natl. Acad. Sci. USA 95:3885-3889, 1998), but not in the thymus of wild type mice (FIG. 2c).

The increased activity of Akt1 bound to Tcl1 is due to Tcl1 binding only to active (phosphorylated at Thr308 and Ser473) Akt1. Alternatively, Tcl1 acts as a cofactor that facilitates the activation of Akt1. To address this question the binding of Tcl1 to kinase inactive Akt1 mutants was examined. FIG. 1d shows that Tcl1 interacts equally well with wild type Akt1 and the Akt1 Thr308/Ala; Ser473/Ala mutant (AA mutant), a mutant that cannot be activated by phosphorylation. In addition, Tcl1 immunoprecipitates equally well with wild type and the kinase dead Akt1 mutant Lys179/Met. This indicates that binding of Tcl1 to Akt1 is independent of Akt1 phosphorylation or activation status.

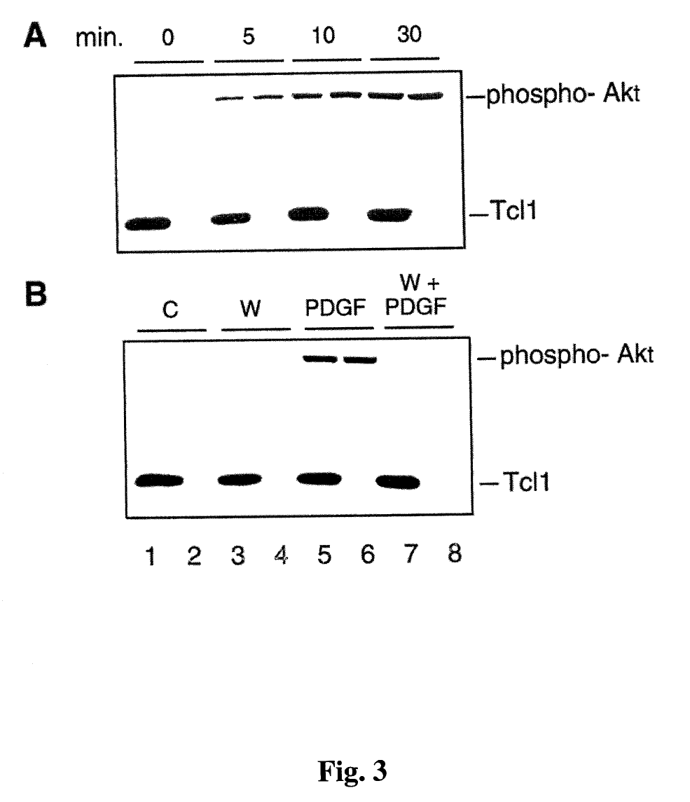

Activation of Akt1 by PDGF is due to D3 phosphoinositides-dependent phosphorylation by PDK1. (Chan, T. O., et al., Annu. Rev. Biochem. 68:965-1014, 1999). Treatment of PDGF-stimulated NIH-3T3 cells with wortmannin, a PI-3K inhibitor, prevents Akt1 phosphorylation and activation. (Bellacosa, A., et al., Oncogene 17:313-325, 1998; Franke, T. F., et al., Cell 81:727-736, 1995). FIG. 3b shows that wortmannin inhibits the phosphorylation of Akt1 in both, untransfected and Tcl1 transfected NIH-3T3 cells. This implies that the stimulatory effect of Tcl1 on the activity of Akt1 is PI-3 kinase dependent and that the binding of Tcl1 to the Akt1 PH domain will not substitute phosphoinositide binding.

The functional outcome of Akt1 phosphorylation is the activation of the Akt1 kinase, therefore a determination of whether overexpression of Tcl1 enhances the phosphorylation of Akt1 at Ser473 by PDGF stimulation was examined. The results show that this is not the case (FIG. 3a). Therefore the effect of Tcl1 on Akt1 activation is PI-3 kinase dependent, but independent of phosphorylation at Ser473. This implies that phosphorylation by PDK1 and binding to Tcl1 may synergise for Akt1 activation.

Tcl1 Promotes Akt1 Nuclear Translocation

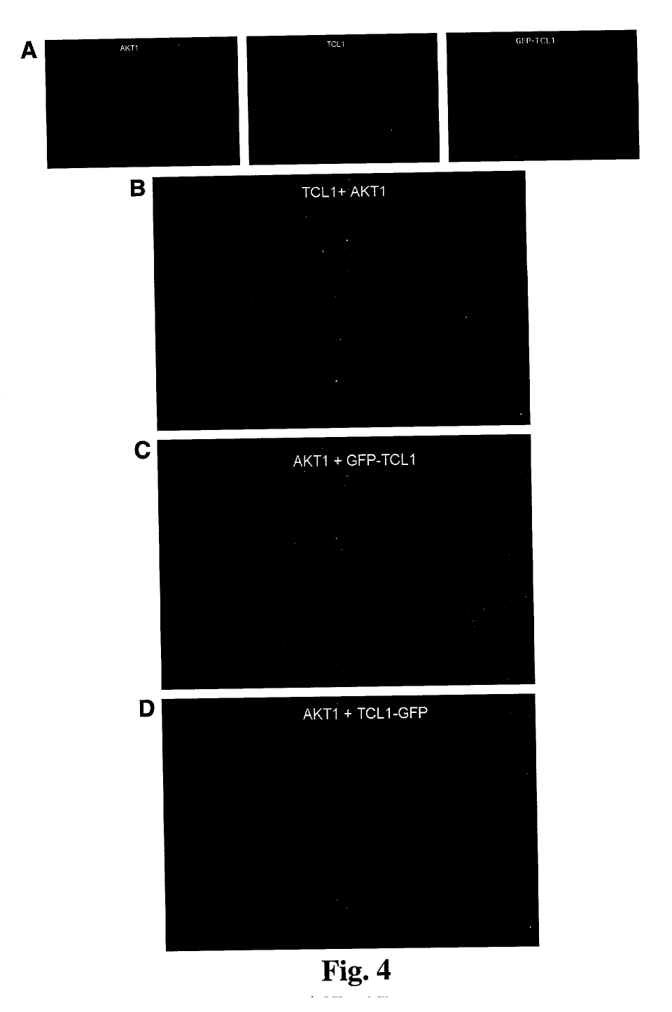

Akt1 is primarily localized in the cytoplasm (Ahmed, N. N., et al., Oncogene 8:1957-63, 1993), although in some cells Akt is localized in the nucleus (Ahmed, N. N., et al., Oncogene 8:1957-63, 1993) and it was reported that in insulin stimulated 293 cells activated Akt1 translocates into the nucleus. (Andjelkovic, M., et al., J. Biol. Chem. 272:31515-31524, 1997). Tcl1, on the other hand, is localized in both, the cytoplasm and in the nucleus. (Fu, T. B., et al., Cancer Res. 54:6297-6301, 1994). Therefore, a determination was made as to whether coexpression of Tcl1 and Akt1 affects the subcellular localization of both proteins. The results of these experiments are shown in FIG. 4. MEF cells were transiently transfected with TCL1 and/or AKT1 and the intracellular localization of both proteins was determined by immunofluorescence. Under normal growth conditions (10% serum) Akt1 was localized in the cytoplasm in more than 90% of cells transfected with AKT1 alone (FIG. 4a, left panel). Under the same growth conditions Tcl1 was localized in both the cytoplasm and the nucleus in more than 90% of cells transfected with TCL1 alone or TCL1-GFP (FIG. 4a, middle and right panels). However, when Tcl1 or a GFP-Tcl1 fusion protein (with GFP attached to the N-terminus of Tcl1) were coexpressed with Akt1 in the same cells, both proteins were colocalized in the cytoplasm as well as in the nucleus in more than 90% of the cells (FIGS. 4b and 4c). Thus, Tcl1 promotes the nuclear translocation of Akt1.

In contrast, coexpression of Tcl1-GFP (with GFP attached to the C-terminus of Tcl1) and Akt1 resulted in localization of Akt1 in the nucleus in only˜30% of the cells. Akt1 was detected mostly in the cytoplasm in the remaining ˜60% of the cells, while Tcl1-GFP remained in its location in the nucleus and in the cytoplasm. (FIG. 4d). This implies that the addition of GFP at the C-terminus of Tcl1, to a certain extend, inhibits the transport of the Tcl1-Akt1 complexes to the nucleus, possibly due to the partial interference with the interaction of Akt1 and Tcl1.

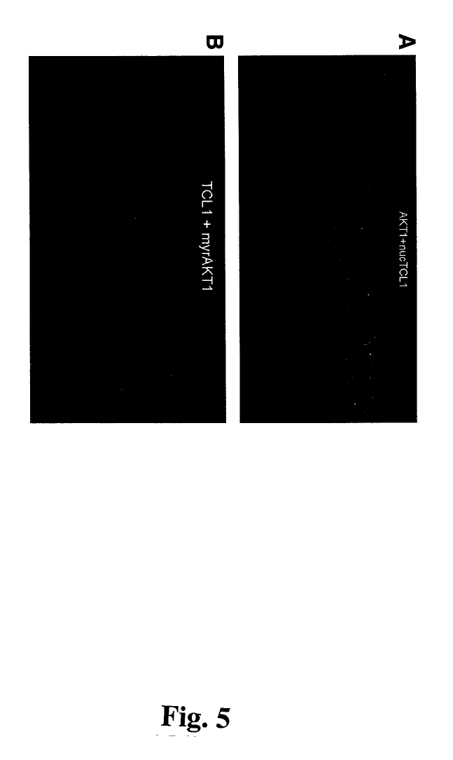

Tcl1 and Akt1 are also localized in the cytoplasm. Thus, the interaction between Tcl1 and Akt1 in the cytoplasm, followed by the translocation of this complex into the nucleus, was examined. A TCL1 construct containing a nuclear localization signal results in the expression of Tcl1 only in the nucleus (nucTcl1). FIG. 5a shows that in cells expressing nuclear Tcl1, Akt1 was located exclusively in the cytoplasm. This implies that Akt1 needs to interact with Tcl1 in the cytoplasm in order to be transported to the nucleus. While interaction of Tcl1 with wild type Akt1 led to the nuclear translocation of Akt1, interaction of Tcl1 with membrane associated myrAkt1 led to the cytoplasmic localization of Tcl1 (FIG. 5b). These results indicate that indeed the binding between the two proteins affects the subcellular localization of both. The nuclear translocation of wild type Akt1 in cells coexpressing both proteins appears to be biologically relevant.

The biological consequences of the enhancement of the Akt1 activity have not been determined to date. However, data indicate that expression of Tcl1 does not increase the Akt1-mediated phosphorylation of Bad, p70 S6 kinase, or IKB. (Mok, C. L., et al., J. Exp. Med. 189:575-86, 1999; Ozes, O. N., et al., Nature 401:82-85, 1999; Pullen, N., et al., Science 279:707-10, 1998).

Discussion

The present invention relates to the physical interaction between Akt1 and Tcl1 and resulting enhancement of the Akt1 kinase activity, as well as the translocation of Akt1 kinase into the nucleus. Although Akt1 and Akt2 are closely related proteins, the data indicate that Tcl1 interacts specifically with Akt1. Furthermore, neither Akt1 nor Akt2 interacted with the Tcl1 related protein, Tcl1b.

The process of Akt activation consists of three distinct steps: 1) a PH-domain dependent, growth factor independent step, marked by constitutive phosphorylation of Thr450; 2) a growth factor induced PI-3K dependent membrane translocation step; and 3) a PI-3K dependent step characterized by phosphorylation at Thr308 and Ser473. (Bellacosa, A., et al., Oncogene 17:313-325, 1998). Both PI-3K dependent steps are inhibited by wortmannin, a PI-3K inhibitor. (Franke, T. F., et al., Cell 81:727-736, 1995). The data disclosed herein revealed that Tcl1 does not activate Akt1 in wortmannin treated cells; therefore, binding of Tcl1 to the Akt1 PH domain can not substitute for D3-phosphoinositide binding. Moreover, Tcl1 does not enhance Akt1 phosphorylation, implying that binding of Tcl1 to Akt1 will act in conjunction with phosphorylation to induce activation of Akt1. Alternatively, the Tcl1-Akt1 complex will recruit additional proteins which enhance the activity of Akt1.

Recent studies showed that Akt1 can be found in the nucleus (Ahmed, N. N., et al., Oncogene 8:1957-63, 1993) and in insulin stimulated 293 cells nuclear translocation of Akt1 will take place following its membrane translocation and activation. (Andjelkovic, M., et al., J. Biol. Chem. 272:31515-31524, 1997). The data disclosed herein provide one mechanism of nuclear translocation of Akt1, specifically in MEF cells grown under normal conditions and coexpressing Akt1 and Tcl1, the Akt1 was constitutively localized in the nucleus. The change in the subcellular localization of Akt1 is dependent on the interaction between the two proteins. This is further supported by data showing that membrane-associated myrAkt1 forces Tcl1 into the cytoplasm. The interaction between Akt1 and Tcl1 responsible for the nuclear translocation of Akt1 appears to occur in the cytoplasm. These data imply that Tcl1 not only facilitates the activation of Akt1, but also promotes its nuclear translocation. The latter may be due to the fact that Tcl1 functions as a direct transporter of Akt1 or contributes to the assembly of a complex that promotes the nuclear transport of Akt1. Since Tcl1 is expressed only in certain lymphoid cells (Virgilio, L., et al., Proc. Natl. Acad. Sci. USA 91:12530-12534, 1994), and the nuclear translocation of Akt1 was reported in cells not expressing Tcl1 (Andjelkovic, M., et al., J. Biol. Chem. 272:31515-31524, 1997), additional molecules, perhaps related to Tcl1, responsible for Akt1 nuclear translocation may exist.

The biological outcome of the Tcl1-induced enhancement of Akt1 activity is expected to occur through the phosphorylation of Akt1 specific targets. Since the Tcl1-activated Akt1 translocates into the nucleus, the most likely targets of the Tcl1-Akt1 complex are nuclear. To address these questions, phosphorylation of previously reported cytoplasmic proteins were examined for their ability to be phosphorylated by Akt1, either directly or indirectly. The results to date imply that Tcl1 does not enhance the Akt1-mediated phosphorylation of p70 S6 kinase, Bad and IKB. Future studies will investigate the phosphorylation of nuclear targets.

Since both Tcl1 and Akt1 cause T-cell malignancies in transgenic mice, it will be of considerable interest to determine whether TCL1 and AKT1 double transgenic mice develop leukemia faster or show a more severe phenotype. In summary, the participation of Tcl1 in the PI-3 kinase dependent Akt1 signaling pathway enhances Akt1 kinase activity and mediates Akt1 nuclear translocation. The present invention relates to the inhibition of Tcl1 binding to Akt1, thus precluding the formation of a Tcl1-Akt1 complex and subsequent enhancement of Akt1 kinase activity.

Monoclonal Antibodies to Antigenic Epitopes on Tcl1

Methods to prepare and isolate monoclonal antibodies to known antigenic epitopes are well known to those skilled in the art. Materials and methods are described in Harlow, E. and Lane, D, Antibody Laboratory Manual, Cold Spring Harbor Press, pages 139-245, 1998, which is incorporated herein by reference. Monoclonal antibodies are isolated and 20-50 μg of each monoclonal antibody is mixed with 1-10 μg, preferably 5 μg, Akt1 or the PH domain of Akt1 and 1-10 μg, preferably 5 μg of Tcl1 in lysis buffer (Protein lysates, immunopreciptiation, and Western blotting, supra), with total reaction volume of 500 μl. Following incubation at 37° C. overnight, each monoclonal antibody reaction is immunoprecipitated, as described supra, with anti-Tcl1 clone 27D6 mouse monoclonal antibody. The presence of Akt1 in each Tcl1 immunoprecipitate is tested by Western blotting, performed under standard conditions (Fu, T. B., et al., Cancer Res. 54:6297-6301, 1994), supra. The absence of Akt1 in the Tcl1 immunopreciptates identifies the monoclonal antibodies that bind to the Tcl1 epitopes responsible for the interaction with Akt1, thereby inhibiting the Tcl1-Akt1 complex formation. The present invention relates to the modulation of Tcl1 enhanced kinase activity by inhibiting Tcl1-Akt1 complex formation, particularly to therapeutic or pharmaceutical compositions containing these antibodies, as described infra.

Inhibition of Tcl1-Akt1 Complex Formation by the PH Domain Fragment of Akt1 Kinase

Tcl1 binds to the PH domain of Akt1 kinase; therefore, a peptide fragment of the Akt1 kinase PH domain will modulate the formation of a Tcl1-Akt1 complex. Aberrant Tcl1 expression occurs in chromosomal abnormalities at the 14q32.1 locus and is observed in several types of T-cell leukemias and lymphomas (Virgilio., et al., Proc. Natl. Acad. Sci. USA 91: 12530-12534, 1994; Narducci, M. G., et al., Cancer Res. 57:5452-5456, 1997). One function of Tcl1 is to bind to the PH domain of Akt1 kinase and enhance its activity, promoting cell cycle progression and thus proliferation. Since this aberrant Akt1 kinase activity causes unregulated cell cycle progression, and thereby facilitates the development of T-cell lymphomas, inhibiting the formation of the Tcl1-Akt1 kinase complex will preclude any Tcl1 enhanced proliferative effect. The present invention relates to the expression of a peptide fragment of the Akt1 kinase, specifically the PH domain, in cells, its binding to Tcl1, and inhibition of any Tcl1-Akt1 kinase complex.

NIH-3T3, 293 and SupT11 cells are transfected with constructs containing Akt1 kinase or vector only. Endogenous Akt1 is immunoprecipitated 48 hours later from lysates of transfected cells using anti-Tcl1 or anti-Akt1 antibodies. The kinase activity associated with these immune complexes is measured, as described supra.

Aberrant cell proliferation is an effect of enhanced Akt1 kinase activity, which occurs when Tcl1 binds to the PH domain of the Akt1 kinase. Inhibition of this Tcl1 enhanced activity will be further pursued in vivo for inhibition of aberrant cell proliferation induced by aberrant TCL1 expression, as occurs in 14q32.1 abnormalities. Since mature T-cells in circulation do not express TCL1 unless they are activated, as in T-cell leukemias and lymphomas, preventing Tcl1 from binding to Akt1 kinase will preclude any subsequent enhancement of Akt1 kinase induced proliferation.

Retroviral vectors or other vectors such as adenotvirus or adeno-associated viral vectors are well known to those skilled in the art, see for example U.S. Pat. No. 4,980,286. An appropriate nucleic acid expression vector that encodes the PH domain of Akt1 kinase is constructed. The present invention relates to therapeutic or pharmaceutical compositions of PH domain expressing retroviral vectors, as described infra.

Therapeutic compositions containing the PH domain retroviral vectors are administered to TCL1 transgenic mice, mice that develop mature leukemia after only 15 months (Virgilio, L., et al., Proc. Natl. Acad. Sci USA, 95: 3885-3889, 1998; Gritti, C., et al., Blood, 92: 368-373, 1998). The in vivo therapeutic efficacy is monitored in this model system by the absence of development of mature leukemia.

Screening for Tcl1 Mimics and Antagonists

The present invention relates to the detection of molecules that specifically bind to Akt1 kinase and thereby modify its activity. Such molecules will thus affect cell proliferation. In a preferred embodiment, assays are performed to screen for molecules with potential utility as therapeutic agents or lead compounds for drug development. The invention provides assays to detect molecules that mimic Tcl1, thereby activating the Tcl1 enhanced activation of Akt1 kinase and promoting cell proliferation. The invention further provides assays to detect molecules that antagonize Tcl1's effect on Akt1 kinase, thereby inhibiting activation of Akt1 kinase and subsequent cell proliferation while promoting programmed cell death (apoptosis).

For example, recombinant cells expressing Akt1 kinase nucleic acids are used to recombinantly produce Akt1 kinase and screen for molecules that bind to Akt1 kinase. Molecules are contacted with the Akt1 kinase, or fragment thereof, under conditions conducive to binding, and then molecules that specifically bind to the Akt1 kinase are identified. Methods that are used to carry out the foregoing are commonly known in the art.

In a specific embodiment of the present invention, an Akt1 kinase and/or cell line that expresses an Akt1 kinase is used to screen for antibodies, peptides, or other molecules that bind to the Akt1 kinase and act as a Tcl1 mimic or antagonist of Tcl1. While Tcl1 is expressed in cells of the lymphoid line, the Tcl1 mimics and antagonists of the present invention will function in any cell. Tcl1 mimics will activate the Tcl1 enhanced activation of Akt1 kinase, thereby promoting a cell proliferative response. Therefore, Tcl1 mimics of the present invention will inhibit or prevent a disease state associated with excessive cell death, as occurs in degenerative diseases. Such disease states include, but are not limited to, Alzheimer's, Armanni-Ehrlich's, macular degenerative diseases, etc.

In contrast, Tcl1 antagonists will modulate the activity of Akt1 kinase and are used to inhibit or prevent a disease state associated with cell overproliferation. Such disease states include, but are not limited to, leukemias, lymphomas and other cancers, restenosis, etc.

Tcl1 mimics and antagonists are identified by screening organic or peptide libraries with recombinantly expressed Akt1 kinase. These Tcl1 mimics and antagonists are useful as therapeutic molecules, or lead compounds for the development of therapeutic molecules, to modify the activity of Akt1 kinase. Synthetic and naturally occurring products are screened in a number of ways deemed routine to those of skill in the art.

By way of example, diversity libraries, such as random or combinatorial peptide or nonpeptide libraries are screened for molecules that specifically bind to Akt1 kinase. Many libraries are known in the art that are used, e.g., chemically synthesized libraries, recombinant (e.g., phage display libraries), and in vitro translation-based libraries.

Examples of chemically synthesized libraries are described in (Fodor et al., Science 251:767-773, 1991; Houghten et al., Nature 354:8486, 1991; Lam et al., Nature 354:82-84, 1991; Medynski, Bio/Technology 12:709-710, 1994; Gallop et al., J. Medicinal Chemistry 37(9):1233-1251, 1994; Ohlmeyer et al., Proc. Natl. Acad. Sci. USA 90:10922-10926, 1993; Erb et al., Proc. Natl. Acad. Sci. USA 91:11422-11426, 1994; Houghten et al., Biotechniques 13:412, 1992; Jayawickreme et al., Proc. Natl. Acad. Sci. USA 91:1614-1618, 1994; Salmon et al., Proc. Natl. Acad. Sci. USA 90:11708-11712, 1993; PCT Publication No. WO 93/20242; and Brenner and Lerner, Proc. Natl. Acad. Sci. USA 89:5381-5383, 1992).

Examples of phage display libraries are described in (Scott and Smith, Science 249:386-390, 1990; Devlin et al., Science, 249:404-406, 1990; Christian, R. B., et al., J. Mol. Biol. 227:711-718, 1992; Lenstra, J. Immunol. Meth. 152:149-157, 1992; Kay et al., Gene 128:59-65, 1993; and PCT Publication No. WO 94/18318 dated Aug. 18, 1994).

In vitro translation-based libraries include, but are not limited to, those described in (PCT Publication No. WO 91/0505 dated Apr. 18, 1991; and Mattheakis et al., Proc. Natl. Acad. Sci. USA 91:9022-9026, 1994).

By way of examples of nonpeptide libraries, a benzodiazepine library (see e.g., Bunin et al., Proc. Natl. Acad. Sci. USA 91:4708-4712, 1994) can be adapted for use. Peptoid libraries (Simon et al., Proc. Natl. Acad. Sci. USA 89:9367-9371, 1992) can also be used. Another example of a library that can be used, in which the amide functionalities in peptides have been permethylated to generate a chemically transformed combinatorial library, is described by (Ostresh et al., Proc. Natl. Acad. Sci. USA 91:11138-11142, 1994).

Screening the libraries is accomplished by any of a variety of commonly known methods. See, e.g., the following references, which disclose screening of peptide libraries: (Parmley and Smith, Adv. Exp. Med. Biol. 251:215-218, 1989; Scott and Smith, Science 249:386-390, 1990; Fowlkes et al., BioTechniques 13:422-427, 1992; Oldenburg et al., Proc. Natl. Acad. Sci. USA 89:5393-5397, 1992; Yu et al., Cell 76:933-945, 1994; Staudt et al., Science 241:577-580, 1988; Bock et al., Nature 355:564-566, 1992; Tuerk et al., Proc. Natl. Acad. Sci. USA 89:6988-6992, 1992; Ellington et al., Nature 355:850-852, 1992; U.S. Pat. No. 5,096,815, U.S. Pat. No. 5,223,409, and U.S. Pat. No. 5,198,346, all to Ladner et al.; Rebar and Pabo, Science 263:671-673, 1993; and PCT Publication No. WO 94/18318).

In a specific embodiment, screening is carried out by contacting the library members with Akt1 kinase, or fragment thereof, immobilized on a solid phase and harvesting those library members that bind to the Akt1 kinase, or fragment thereof. Examples of such screening methods, termed “panning” techniques are described by way of example in (Parmley and Smith, Gene 73:305-318, 1988; Fowlkes et al., BioTechniques 13:422-427, 1992; PCT Publication No. WO 94/18318) and in references cited hereinabove.

In another embodiment, the two-hybrid system for selecting interacting proteins in yeast (Fields and Song, Nature 340:245-246, 1989; Chien et al., Proc. Natl. Acad. Sci. USA 88:9578-9582, 1991) is used to identify molecules that specifically bind to Akt1 kinase, or fragment thereof

Therapeutic Utility

The monoclonal antibodies, viral vectors, and Tcl1 mimics and antagoists of the present invention are tested in vivo for the desired therapeutic or prophylactic activity. For example, such compounds are tested in suitable animal model systems prior to testing in humans, including but not limited to rats, mice, chicken, cows, monkeys, rabbits, etc. For in vivo testing, prior to administration to humans, any animal model system known in the art may be used.

Therapeutic/Prophylactic Methods and Compositions

The invention provides methods of treatment and prophylaxis by administration to a subject an effective amount of a therapeutic, i.e., a monoclonal (or polyclonal) antibody, viral vector, Tcl1 mimic or Tcl1 antagonist of the present invention. In a preferred aspect, the therapeutic is substantially purified. The subject is preferably an animal, including but not limited to, animals such as cows, pigs, chickens, etc., and is preferably a mammal, and most preferably human.

Various delivery systems are known and are used to administer a therapeutic of the invention, e.g., encapsulation in liposomes, microparticles, microcapsules, expression by recombinant cells, receptor-mediated endocytosis (see, e.g., Wu and Wu, J. Biol. Chem. 262:4429-4432, 1987), construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. U.S.A. 88:1864-1868, 1991), etc. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10 k by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

What is claimed is:

1. An antibody which binds to an epitope on Tcl1, wherein said antibody modulates an interaction between said epitope and an Akt1 kinase.

2. The antibody of claim 1, wherein said antibody modulates a Tcl1 enhanced kinase activity of said Akt1 kinase.

3. The antibody of claim 1, wherein said antibody comprises a monoclonal antibody.

4. The antibody of claim 1, wherein said antibody comprises a polyclonal antibody.

5. A pharmaceutical composition comprising an antibody of claim 1.

6. A method of treating a disease state in which the activity of an Akt1 kinase is altered in a mammal, comprising administering to said mammal a therapeutically effective amount of said antibody of claim 1, wherein said antibody binds to an epitope on a Tcl1 protein, thereby modulating a Tcl1 enhanced kinase activity of said Akt1 kinase.

7. The method of claim 6, wherein said disease state is a T-cell leukemia or T-cell lymphoma.

8. The method of claim 7, wherein said T-cell leukemia or T-cell lymphoma is associated with a chromosome 14 abnormality, said chromosome abnormality further comprises a t(14;14) (q11;q32) translocation or an inv (14) (q11;132) inversion.

9. A method of treating a disease state in which the activity of an Akt1 kinase is altered in a mammal, comprising administering to said mammal a therapeutically effective amount of a peptide fragment of an Akt1 kinase, said peptide fragment further comprising a PH domain, wherein said peptide fragment binds to said Akt kinase, thereby modulating a Tcl1enhanced kinase activity of said Akt1 kinase.

10. The method of claim 9, wherein said disease state is a T-cell leukemia or T-cell lymphoma.

11. The method of claim 10, wherein said T-cell leukemia or T-cell lymphoma is associated with a chromosome 14 abnormality, said chromosome abnormality further comprising a t(14;14) (q11;q32) translocation or an inv (14) (q11;132) inversion.

12. The method of claim 10, wherein said peptide fragment comprises a PH domain, wherein said PH domain competitively binds to an Akt1 binding domain on a Tcl1 protein.

13. A pharmaceutical composition comprising a peptide fragment of an Akt1 kinase, said peptide fragment comprising a PH domain.

14. A compound comprising a Tcl1 mimic, wherein said Tcl1 mimic binds to an Akt1 kinase in any cell and is functionally active in mimicking a Tcl1 enhanced activation of said Akt1 kinase.

15. A method of identifying a molecule that specifically binds to an Atk1 kinase and is functionally active in mimicking a Tcl1 enhanced activation of said Akt1 kinase, comprising
a) contacting said Akt1 kinase with a plurality of molecules under conditions conducive to binding between said Akt1 kinase and said molecules; and
b) identifying a molecule within said plurality that specifically binds to said Akt1 kinase and is functionally active in mimicking said Tcl1 enhanced activation.

16. A method of treating a disease state in which the activity of an Akt1 kinase is altered in a mammal, comprising administering to said mammal a therapeutically effective amount of a Tcl1 mimic, wherein said Tcl1 mimic binds to said Akt1 kinase, thereby activating a Tcl1 enhanced kinase activity of said Akt1 kinase.

17. The method of claim 16, wherein said disease state comprises a degenerative disease.

18. A pharmaceutical composition comprising a Tcl1 mimic, wherein said Tcl1 mimic activates a Tcl1 enhanced kinase activity of an Akt1 kinase.

19. A compound comprising a Tcl1 antagonist, wherein said Tcl1 antagonist binds to an Akt1 kinase in any cell and is functionally active in modulating a Tcl1 enhanced activation of said Akt1 kinase.

20. A method of identifying a molecule that specifically binds to an Atk1 kinase and is functionally active in antagonizing a Tcl1 enhanced activation of said Akt1 kinase, comprising
a) contacting said Akt1 kinase with a plurality of molecules under conditions conducive to binding between said Akt1 kinase and said molecules; and
b) identifying a molecule within said plurality that specifically binds to said Akt1 kinase and is functionally active in antagonizing said Tcl1 enhanced activation.

21. A method of treating a disease state in which the activity of an Akt1 kinase is altered in a mammal, comprising administering to said mammal a therapeutically effective amount of a Tcl1 antagonist, wherein said Tcl1 antagonist binds to said Akt1 kinase, thereby inhibiting a Tcl1 enhanced kinase activity of said Akt1 kinase.

22. The method of claim 21, wherein said disease state comprises a proliferative disorder.

23. A pharmaceutical composition comprising a Tcl1 antagonist, wherein said Tcl1 antagonist inhibits a Tcl1 enhanced kinase activity of an Akt1 kinase.
